(19) United States
(12) Patent Application Publication
(10) Pub. No.: US 2025/0090769 A1
(43) Pub. Date: Mar. 20, 2025

(54) CLAMPING DEVICE FOR STEADY NEEDLE INJECTING

(71) Applicant: Shih-Wei Hsieh, Tainan City (TW)

(72) Inventor: Shih-Wei Hsieh, Tainan City (TW)

(21) Appl. No.: 18/469,886

(22) Filed: Sep. 19, 2023

(51) Int. Cl. A61M 5/42

(52) U.S. Cl. CPC A61M 5/427; A61M 2205/3375; A61M 2209/082

(57) ABSTRACT

A clamping device includes a positioning shank, a clamping module pivotally connected with the positioning shank, an ultrasonic searcher clamped by the clamping module, a fixture mounted on the positioning shank, a needle holder pivotally connected with the fixture, and a needle hub mounted on the needle holder. The clamping module includes a positioning frame, multiple abutting pieces mounted in the positioning frame, and multiple push knobs rotatably mounted on the positioning frame and pressing the abutting pieces. The abutting pieces are moved to press the ultrasonic searcher. The push knobs are rotated to move the abutting pieces forward and backward. The abutting pieces are pushed inward by the push knobs so that the clamping module is used to mount ultrasonic searchers of different sizes.

### BACKGROUND OF THE INVENTION

### Field of the Invention

The present invention relates to a clamping device and, more particularly, to a clamping device for steady injecting.

### Description of the Related Art

In the hospital, the medical staff usually needs to perform a needle injection. In general, the blood is transported through contraction of the heart, so the blood vessels fluctuate with the heartbeat and are not obvious on the surface of the patient's skin. Therefore, the medical staff has to find out the location of the blood vessels according to experience, such as by observing the location of the blood vessels with the naked eye, or by sensing the beating of the pulse with the hand. However, when the blood vessels of the patient are too thin and not obvious, or go deep into the subcutaneous tissue, it is difficult for the medical staff to find the location of the blood vessels with the naked eye. Thus, the medical staff is hard to exactly identify the location for the needle injection, which results in the need for multiple needle insertions to successfully insert or inject the needle into the blood vessels so that the patient has to suffer from multiple needle injections.

### BRIEF SUMMARY OF THE INVENTION

In accordance with the present invention, there is provided a clamping device comprising a positioning shank, a clamping module pivotally connected with an end of the positioning shank, an ultrasonic searcher clamped by the clamping module, a fixture mounted on the positioning shank, a needle holder pivotally connected with the fixture, and a needle hub mounted on and inserted into the needle holder. The needle holder extends downward from the fixture. The clamping module includes a positioning frame, multiple abutting pieces mounted in the positioning frame, and multiple push knobs rotatably mounted on the positioning frame and pressing the abutting pieces. The abutting pieces are arranged in every direction in the positioning frame. The abutting pieces are moved to press the ultrasonic searcher. The push knobs are rotated to move the abutting pieces forward and backward. The abutting pieces at a left side, a right side, and an outside of the positioning frame are pushed by the push knobs to move forward and backward. The abutting pieces are pushed inward by the push knobs so that the clamping module is used to mount ultrasonic searchers of different sizes.

In use, the ultrasonic searcher is mounted in the positioning frame. At this time, the abutting pieces are spaced from the ultrasonic searcher. Then, the push knobs are rotated to move the abutting pieces which are pushed inward and moved to press the ultrasonic searcher so that the ultrasonic searcher is fixed on the positioning shank to perform a searching operation. After the position of the blood vessel is determined and confirmed, the needle holder on the fixture is used to position the direction and position of the needle so that the needle aligns with the determined position to perform an injection.

According to the primary advantages of the present invention, the clamping module clamps the ultrasonic searcher so that the blood vessels are exposed exactly, while the angle and direction of the needle holder is set steadily so that the needle is inserted into the blood vessels accurately, thereby achieving a needle injection safely and smoothly.

According to another advantage of the present invention, the ultrasonic searcher uses ultrasonic waves to explore and detect the position and depth of the blood vessel, while the needle of the needle hub is positioned by the needle holder and the fixture, so that the medical staff performs a needle injection stably according to the position of the blood vessel, without having to worry about the mistake or error caused by the medical staff, thereby greatly reducing the psychological pressure of the injection giver and recipient.

According to a further advantage of the present invention, the clamping device is adjusted to clamp ultrasonic searchers of different sizes, while the depth of the blood vessel is located and exposed exactly, so that the needle injection is performed at a time precisely.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a perspective view of a clamping device in accordance with the preferred embodiment of the present invention.

FIG. 2 is a locally enlarged perspective view of the clamping device in accordance with the preferred embodiment of the present invention.

FIG. 3 is a schematic cross-sectional operational view of the clamping device as shown in FIG. 2 in use.

FIG. 4 is a schematic operational view of the clamping device as shown in FIG. 1 in use.

### DETAILED DESCRIPTION OF THE INVENTION

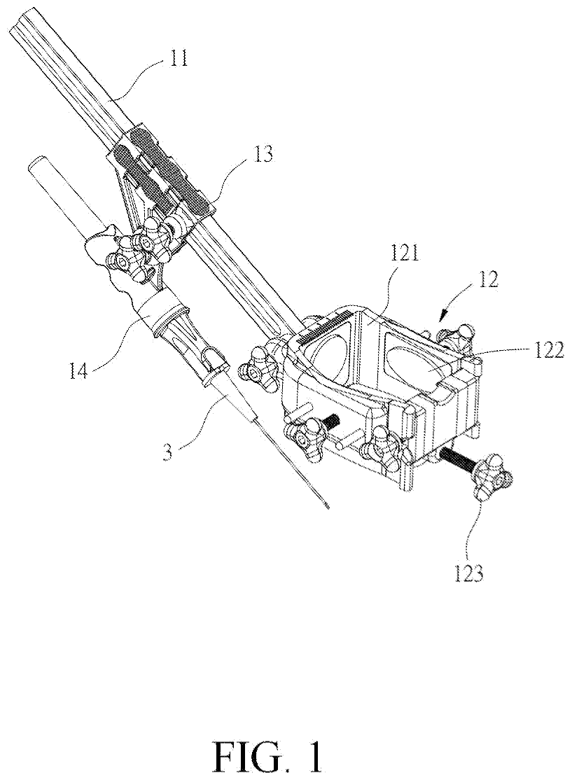

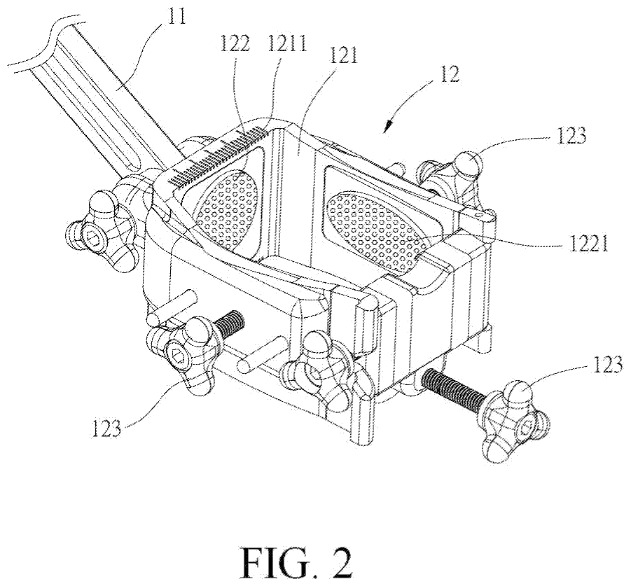

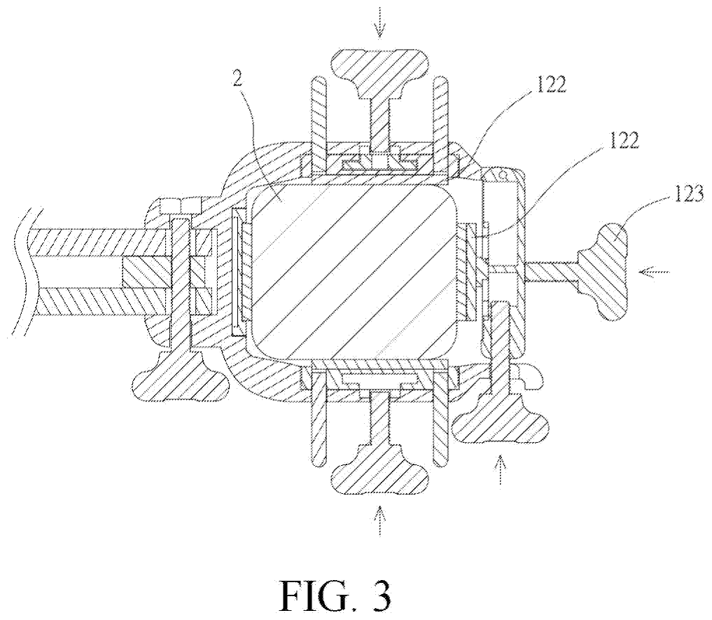

Referring to the drawings and initially to FIGS. 1-3, a clamping device in accordance with the preferred embodiment of the present invention comprises a positioning shank 11, a clamping module 12 pivotally connected with an end of the positioning shank 11, an ultrasonic searcher (or detector or sensor or range finder) 2 clamped by the clamping module 12, a fixture (or holder) 13 mounted on the positioning shank 11, a needle holder 14 pivotally connected with the fixture 13, and a needle hub 3 mounted on and inserted into the needle holder 14.

The needle holder 14 extends downward from the fixture 13. The needle hub 3 has a front end provided with a needle 31. The clamping module 12 includes a positioning frame 121, multiple abutting pieces 122 mounted in the positioning frame 121, and multiple push knobs 123 rotatably mounted on the positioning frame 121 and pressing the abutting pieces 122. The abutting pieces 122 are arranged in every direction (or position) in the positioning frame 121. The abutting pieces 122 are moved to press the ultrasonic searcher 2. The push knobs 123 are rotated to move the abutting pieces 122 forward and backward. The abutting pieces 122 at a left side, a right side, and an outside of the positioning frame 121 are pushed by the push knobs 123 to move forward and backward. Thus, the abutting pieces 122 are pushed inward by the push knobs 123 so that the clamping module 12 is used to mount ultrasonic searchers 2 of different sizes.

In practice, the push knobs 123 are rotated to move the abutting pieces 122 which are pushed inward and moved to press the ultrasonic searcher 2. Thus, the ultrasonic searcher 2 is used to search and detect the blood in the user's skin. In addition, the needle hub 3 on the needle holder 14 is aligned with the position detected by the ultrasonic searcher 2 to perform an accurate needle injection.

In the preferred embodiment of the present invention, the positioning frame 121 of the clamping module 12 is provided with a ruler (or scale) 1211 for measuring a size of the ultrasonic searcher 2.

In the preferred embodiment of the present invention, each of the abutting pieces 122 in the positioning frame 121 of the clamping module 12 is provided with an antiskid layer 1221 pressing the ultrasonic searcher 2 steadily to prevent the ultrasonic searcher 2 from slipping.

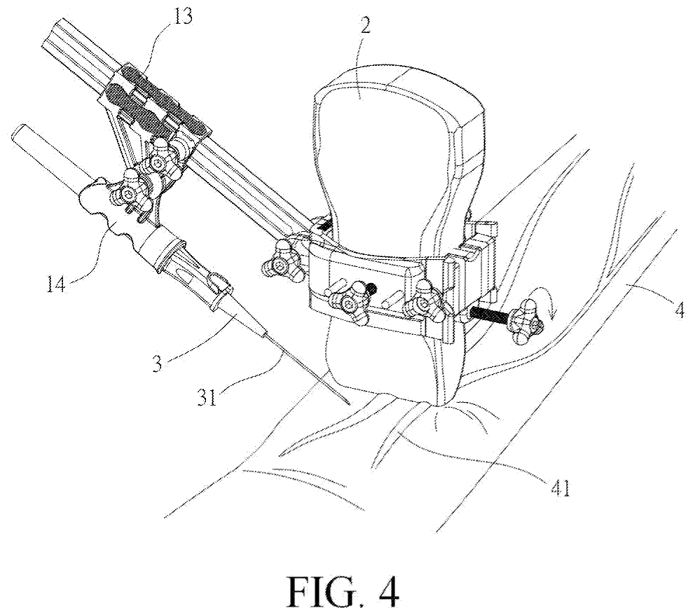

In operation, referring to FIG. 4 with reference to FIGS. 1-3, the ultrasonic searcher 2 is started initially. Then, the ultrasonic searcher 2 is moved to a position to be injected. Then, the ultrasonic searcher 2 is used for searching the user's arm 4 to detect and expose the position of blood vessels 41 in the user's arm 4. At this time, the needle 31 of the needle hub 3 on the needle holder 14 aligns with the blood vessels 41 to perform a needle injection. In such a manner, the operator uses the clamping device to perform an accurate needle injection. Thus, the giver performs the needle injection exactly to reduce errors caused by fatigue, inexperience or shaking hands of the giver, thereby reducing the chance of the recipient getting more times of injection.

Accordingly, the ultrasonic searcher 2 cooperates with the needle holder 14 which holds the needle hub 3 so that the needle hub 3 is kept at a fixed angle, and the needle 31 of the needle hub 3 is directed toward a direction aligning with the position of the blood vessels 41 to facilitate the medical staff performing an accurate needle injection. In addition, the abutting pieces 122 are moved and adjusted to fit the size of the ultrasonic searcher 2 so that the clamping module 12 is used to mount ultrasonic searchers 2 of different sizes.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

1. A clamping device comprising:
a positioning shank;
a clamping module pivotally connected with an end of the positioning shank;
an ultrasonic searcher clamped by the clamping module;
a fixture mounted on the positioning shank;
a needle holder pivotally connected with the fixture; and
a needle hub mounted on and inserted into the needle holder;
wherein:
the needle holder extends downward from the fixture;
the clamping module includes a positioning frame, multiple abutting pieces mounted in the positioning frame, and multiple push knobs rotatably mounted on the positioning frame and pressing the abutting pieces;
the abutting pieces are arranged in every direction in the positioning frame;
the abutting pieces are moved to press the ultrasonic searcher;
the push knobs are rotated to move the abutting pieces forward and backward;
the abutting pieces at a left side, a right side, and an outside of the positioning frame are pushed by the push knobs to move forward and backward; and
the abutting pieces are pushed inward by the push knobs so that the clamping module is used to mount ultrasonic searchers of different sizes.

2. The clamping device as claimed in claim 1, wherein the positioning frame of the clamping module is provided with a ruler for measuring a size of the ultrasonic searcher.

3. The clamping device as claimed in claim 1, wherein each of the abutting pieces in the positioning frame of the clamping module is provided with an antiskid layer pressing the ultrasonic searcher steadily to prevent the ultrasonic searcher from slipping.
